(19) United States
(12) Patent Application Publication
(10) Pub. No.: US 2009/0163978 A1
(43) Pub. Date: Jun. 25, 2009

(54) IMPLANTABLE ELECTRET MICROPHONE

(75) Inventors: Scott Allan Miller, III, Golden, CO (US); Travis Rian Andrews, Loveland, CO (US); Robert Edwin Schneider, Erie, CO (US); David L. Basinger, Loveland, CO (US); James R. Easter, Lyons, CO (US)

Correspondence Address: MARSH, FISCHMANN & BREYFOGLE LLP, 8055 East Tufts Avenue, Suite 450, Denver, CO, 80237, US

(73) Assignee: Otologics, LLC, Boulder, CO (US)

(21) Appl. No.: 12/275,018

(22) Filed: Nov. 20, 2008

Related U.S. Application Data
(60) Provisional application No. 60/989,179, filed on Nov. 20, 2007.

(51) Int. Cl. A61F 11/04

(52) U.S. Cl. 607/57

(57) ABSTRACT

An implantable microphone comprises a hermetically-sealed, enclosed volume and an electret member and back plate disposed with a space therebetween and capacitively coupleable to provide an output signal indicative of acoustic signals incident upon at least one of the electret member and back plate. The back plate may be disposed to define a peripheral portion of the enclosed volume, e.g., the back plate may be defined as part of a flexible diaphragm that receives external acoustic signals. Vents may be provided to fluidly interconnect first and second portions of the enclosed volume that are located on first and second sides of the electret member. In another embodiment, the electret member may be flexible and spaced relative to a flexible outer diaphragm.

### RELATED APPLICATIONS

This application claims priority to U.S. Provisional Application Ser. No. 60/989,179, filed Nov. 20, 2007, entitled “IMPLANTABLE ELECTRET MICROPHONE”, the entirety of which is hereby incorporated by reference.

### FIELD OF THE INVENTION

The present invention relates to the field of implantable hearing instruments, and in particular, to implantable electret microphones employable in fully- and semi-implantable hearing instrument systems.

### BACKGROUND OF THE INVENTION

Traditional hearing aids are placed in a user's ear canal. The devices function to receive and amplify acoustic signals within the ear canal to yield enhanced hearing. In some devices, “behind-the-ear” units have been utilized which comprise a microphone to transduce the acoustic input into an electrical signal, some type of signal processing circuitry to modify the signal appropriate to the individual hearing loss, an output transducer (commonly referred to in the field as a “receiver”) to transduce the processed electrical signal back into acoustic energy, and a battery to supply power to the electrical components.

Increasingly, a number of different types of fully- or semi-implantable hearing instruments have been developed. By way of example, implantable devices include instruments which employ implanted electromechanical transducers for stimulation of the ossicular chain and/or oval window, instruments which utilize implanted exciter coils to electromagnetically stimulate magnets fixed within the middle ear, and instruments which utilize an electrode array inserted into the cochlea to transmit electrical signals for sensing by the auditory nerve.

In these, as well as other implanted devices, acoustic signals are received by an implantable microphone, wherein the acoustic signal is converted to an electrical signal that is employed to generate a signal to drive an actuator that stimulates the ossicular chain and/or oval window or that is applied to selected electrodes of a cochlear electrode array. As may be appreciated, such implantable hearing instrument microphones must necessarily be positioned at a location that facilitates the receipt of acoustic signals and effective signal conversion/transmission. For such purposes, implantable microphones are most typically positioned in a surgical procedure between a patient's skull and skin, at a location rearward and upward of a patient's ear (e.g., in the mastoid region).

Given such positioning, the size and ease of installation of implantable hearing instrument microphones are primary considerations in the further development and acceptance of implantable hearing instrument systems. Further, it is important that a relatively high sensitivity and flat frequency response be provided to yield a high fidelity signal. Relatedly, the componentry cost of providing such a signal is of importance to achieving widespread use of implantable systems.

### SUMMARY OF THE INVENTION

In view of the foregoing, a primary objective of the present invention is to provide an implantable microphone having a relatively small profile.

An additional objective of the present invention is to provide an implantable microphone that is reliable and cost effective.

Yet further objectives of the present invention are to provide an implantable microphone that provides high-sensitivity and relatively flat frequency response in acoustic signal conversion.

One or more of the above-noted objectives and additional advantages are realized by an implantable microphone of the present invention. The implantable microphone includes a hermetically-sealed, enclosed volume, and an electret member and back plate disposed with a space therebetween within the enclosed volume. The electret member and back pate are capacitively coupleable to provide an output signal indicative of acoustic signals incident upon at least one of the electret member and back plate. The electret arrangement yields a compact, and relatively low cost arrangement, while also providing a high quality output signal for use by an implantable hearing instrument.

As employed herein, an “electret member” is meant to refer to a microphone component having a dielectric material portion with a permanently-embedded static electric charge and an electrically-conductive material portion, or electrode. Further, a “back plate” is meant to refer to a microphone component having an electrically-conductive material portion, or electrode. When employed together in a microphone, the electret member and back plate may be disposed with the dielectric material portion of the electret member and the electrically-conductive material portion of the back plate located in opposing spaced relation and capacitively coupled, and with at least one of the electret member and back plate being moveable in response to acoustic signals incident thereupon, wherein electrical outputs from the electret member and back plate (e.g. from each of the electrodes) may be utilized to provide an electret output signal.

By way of example only, in a common source configuration, the electret member and back plate may be interconnected to a preamplifer (e.g., a FET) that is powered by a separate power source (e.g., an implantable, rechargeable battery). In turn, the preamplifier output may provide the electret output signal. The electret output signal may be processed and/or otherwise utilized to generate a drive signal applied to a transducer to stimulate a middle ear and/or inner ear component of a patient.

In one aspect, the back plate of the implantable microphone may be disposed so as to define at least a peripheral portion of the enclosed volume. For example, the back plate may be defined as a part of a flexible diaphragm that extends across a housing aperture for receiving external acoustic signals (e.g., transcutaneous signals emanating from outside the body and generating acoustic signals within the enclosed volume in response thereto).

In another aspect, a first portion of the enclosed volume of the implantable microphone may be located on a first side of the electret member and a second portion thereof may be located on a second side of the electret member. In turn, at least one vent may fluidly interconnect the first and second portions, thereby yielding enhanced sensitivity.

In one approach, the vent(s) may extend through the electret member. For example, a plurality of vents may extend through the electret member to fluidly interconnect the first and second portions of the hermetically-sealed, enclosed volume. In such an embodiment, the vents may be spaced in a symmetric manner about a center axis of the electret member.

In a further aspect, the implantable microphone may include a flexible, biocompatible diaphragm that defines a peripheral portion of the enclosed volume. Relatedly, the electret member may be spaced from the diaphragm and be of a flexible construction, wherein the output signal is indicative of acoustic signals that are generated by the diaphragm and incident upon the flexible electret member within enclosed volume of the microphone.

In such an arrangement, a first portion of the enclosed volume may be located on a first side of the back plate and a second portion of the enclosed volume may be located on a second side of the back plate. In turn, at least one vent may be provided through the back plate to fluidly interconnect the first and second portions. In one embodiment, a plurality of vents may extend through the back plate to fluidly interconnect the first and second portions. For example, the plurality of vents may be spaced in a symmetric manner about a center axis of the electret member.

In certain embodiments, the electret member may be provided so that the dielectric material displays a low surface conductance, e.g. a surface resistance of at least about 10 gigaohms, and preferably at least about 100 gigaohms. Additionally, the electret member and back plate may be provided to yield a capacitive coupling therebetween of at least 1 picofarad, and preferably at least 5 picofarad.

In yet another aspect, at least one of the electret member and the back plate may comprise a carrier, or support member. In this regard, the support member may be integrally defined by or separate from the electrically-conductive material portion and/or the dielectric material portion of the electret member, and/or integrally defined by or separate from the electrically conductive material portion of the back plate. For example, a dielectric material and/or electrically conductive material may be supportably disposed upon a support member (e.g. in layers applied thereto).

In some approaches, the electret member may be defined by applying a layer of electrically-conductive material (e.g. via a metallization process) on to a support substrate (e.g. a printed circuit board), and by applying a layer dielectric material (e.g. a Teflon-based material or glass) on to the support substrate or the electrically conductive layer (e.g. via a process in which the dielectric material is applied in a viscous or particulate state and then cured or dried). Similar techniques may be employed to define the electrically-conductive portion of the back plate. As may be appreciated, such approaches may facilitate the provision of an electret member and/or back plate having a desired thickness and/or profile.

In another aspect, the electret member may be defined by applying a dielectric material on to an electrically-conductive support member or on to a separate support member, and charging the dielectric material. In one embodiment, the charging step may occur at least partially contemporaneously with the applying step. For example, the dielectric material may be disposed via radio frequency (RF) sputtering to simultaneously complete the applying and charging steps.

In other embodiments, the dielectric material may be applied to an electrically-conductive support member or a separate support member via spraying, dipping, coating or chemical vapor deposition. In turn, the dielectric material may be charged by heating the dielectric material to a predetermined temperature (e.g. at or above a corresponding Curie temperature), applying a voltage to the heated material (e.g. at or above the corresponding Curie temperature), and then cooling the material. Alternatively, ion implantation and/or charged particle (e.g. bipolar or monopolar particles) corona spray techniques may be employed.

In a related aspect, the back plate may be advantageously positioned relative to a support member of the electret member prior to or immediately after charging of the dielectric material of the electret member, thereby enhancing maintenance of the static charge imparted to the electret member. For example, in one approach the electret member and back plate may be preassembled prior to charging the electret member, then charged and assembled with the balance of the implantable microphone componentry.

Additional aspects and corresponding advantages will be apparent to those skilled it the art upon consideration of the further description that follows.

### BRIEF DESCRIPTION OF THE DRAWINGS

FIG. 1 illustrates a cross-sectional side view of one embodiment of an implantable microphone of the present invention.

FIG. 2 illustrates a cross-sectional side view of one detailed assembly of the embodiment of FIG. 1.

FIG. 3 illustrates an exploded assembly view corresponding with the assembly of FIG. 2.

FIG. 4 illustrates a cross-sectional side view of another embodiment of an implantable microphone of the present invention.

FIG. 5 illustrates a cross-sectional side view of another embodiment of an implantable microphone of the present invention.

FIG. 6 illustrates a cross-sectional side view of another embodiment of an implantable microphone of the present invention.

FIG. 7 illustrates a cross-sectional side view of another embodiment of an implantable microphone of the present invention.

FIG. 8 illustrates a cross-sectional side view of another embodiment of an implantable microphone of the present invention.

FIG. 9 illustrates a cross-sectional side view of another embodiment of an implantable microphone of the present invention.

FIG. 10 illustrates a cross-sectional side view of another embodiment of an implantable microphone of the present invention.

FIG. 11 illustrates a cross-sectional side view of another embodiment of an implantable microphone of the present invention.

### DETAILED DESCRIPTION OF THE INVENTION

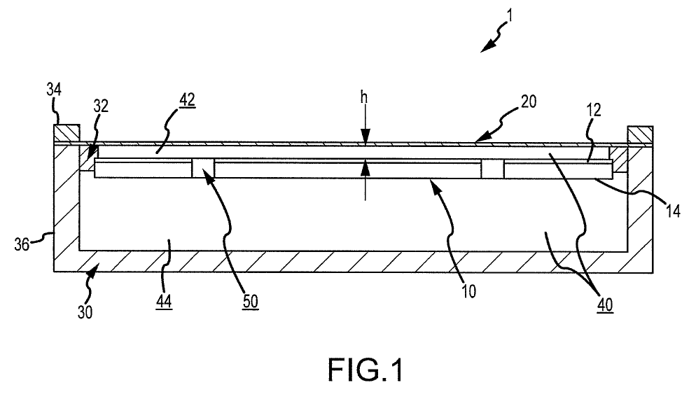

FIG. 1 illustrates one embodiment of the present invention. The implantable microphone 1 includes an electret member 10 and a flexible diaphragm 20 which comprises a back plate. The flexible diaphragm 20 extends across an opening of a biocompatible housing 30 and is peripherally secured in such position between a clamp ring 34 and interconnected (e.g. via laser welding), cup-shaped lower housing member 36. The diaphragm 20 and housing 30 define a hermetically-sealed, enclosed volume 40 that includes a first portion 42 located on a first side of the electret member 10 and a second portion 44 located on an opposing second side of the electret member 10. The first portion 42 and second portion 44 are fluidly interconnected by one or more vents 50 that extend through the electret member 10.

As shown in FIG. 1, the electret member 10 and the diaphragm 20 comprising the back plate may be spaced by a relatively small distance h that comprises the enclosed volume 40. In turn, the electret member 10 and back plate of diaphragm 20 may be capacitively coupleable to provide an output signal indicative of the external acoustic signals incident upon the flexible diaphragm 20.

By way of example only, in a common source configuration, the electret member 10 and back plate of the diaphragm 20 may each be electrically interconnected to a preamplifier (e.g., a FET) that is powered by a separate power source (e.g., an implantable, rechargeable battery). In turn, the preamplifier output may provide an electret output signal. In turn, such output signal may be utilized to generate a drive signal for an implanted hearing aid instrument (e.g., an electromechanical or electromagnetic transducer for middle ear stimulation or a cochlear electrode array).

The electret member 10 may be of a non-flexible construction and disposed in fixed relation to the housing 30. Further, the electret member 10 may be electrically insulated from the housing 30 and back plate of flexible diaphragm 20 by one or more peripheral insulating member(s) 32. Such, peripheral member(s) 32, or other components, may also be disposed to engage and thereby facilitate positioning and tensioning of the diaphragm 20 at a desired distance h from the electret member 10, as shown in FIG. 1, and further discussed below.

The electret member 10 may comprise a charged dielectric material layer 12 and an electrode 14 (e.g., a metal plate or metallized support member). By way of example, the dielectric material layer 12 may comprise a permanently-charged, halocarbon polymer such as polyfluoroethylenepropylene. The diaphragm 20 may comprise an electrically-conductive material, e.g., a biocompatible metal such as titanium, wherein the diaphragm 20 may integrally define the back plate. In other arrangements, a separate metal layer defining the electrode of the back plate may be provided on an internal side of the diaphragm 20.

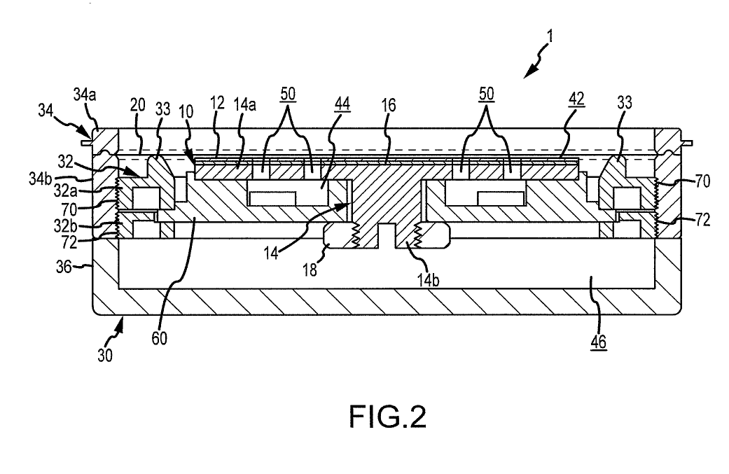

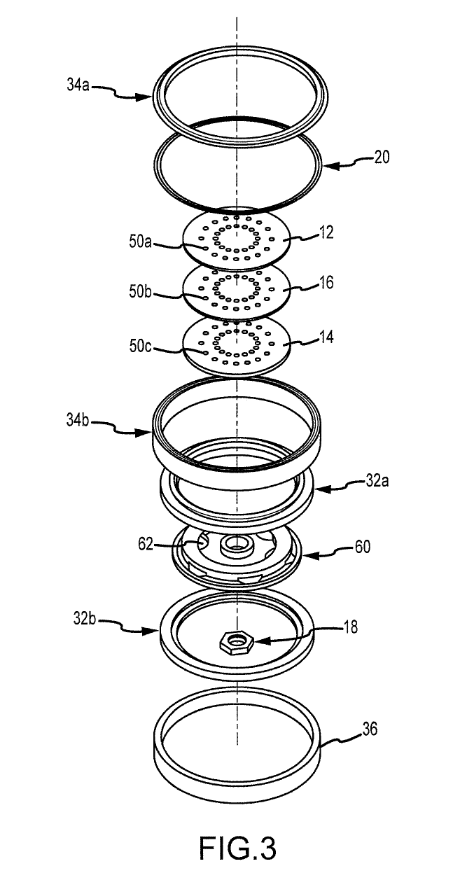

Referring now to FIGS. 2 and 3, a detailed embodiment generally corresponding with the embodiment of FIG. 1 of the present invention is illustrated, wherein corresponding components are referred to with corresponding reference numerals. As illustrated, the implantable microphone 1 includes an electret member 10 comprising a dielectric layer 12 (e.g., a flat circular-shaped Teflon disc) physically interconnected to an underlying electrode 14 (e.g., a T-shaped metal member (e.g. brass) having a circular top plate portion) by an interconnection layer 16 (e.g., a VHB, double adhesive-sided, circular disc). In other arrangements a virgin Teflon may be disposed upon an ultron support member to define a dielectric layer.

In one implementation, one side of an interconnection layer 16 may be adhesively interconnected to a T-shaped electrode 14, and a dielectric layer 12 may be adhesively interconnected to another side of the interconnection layer 16, wherein, the T-shaped electrode 14 supports the dielectric layer 12 and an interconnection layer 16 on a top portion 14a thereof, and further provides a bottom leg portion 14b for advantageously handling the electret member 10 free from user contact with an exposed top surface of the dielectric layer 12 during assembly.

The dielectric layer 12, electrode 14 and interconnection layer 16 may have interfacing portions of a coincidental configuration as illustrated in FIG. 3. Further, the dielectric layer 12, interconnection layer 16 and electrode 14 may each comprise a corresponding plurality of vents 50a, 50b and 50c, respectively, extending therethrough, wherein when such components are disposed in a stacked, laminate fashion, the vents 50a, 50b and 50c are aligned to fluidly interconnect a first portion 42 and second portion 44 of an enclosed volume 40.

In the latter regard, and as is best shown in FIG. 2, at least a part of the second portion 44 may be defined by an annular, recessed ring portion of a mount member 60 that peripherally, supportably receives and positions the electret member 10. The mount member 60 may be electrically non-conductive. The leg portion 14b of a T-shaped electrode 14 may be disposed to extend through an opening of the mount member 60 and be retained in fixed relation thereto by a locking member 18. In turn, the mount member 60 may be peripherally supported by a first peripheral member 32b which peripherally engages and is thereby supported by a housing 30. Further, a second peripheral member 32a may be peripherally provided in opposing relation to the first peripheral member 32b to facilitate positioning of the mount member 60, as well as tensioning of diaphragm 20 relative to the electret member 10. As may be appreciated, the mount member 60 and/or first peripheral support member 32b and/or second peripheral support member 32a may comprise an electrically non-conductive material so as to electrically insulate the electrode 14 from the housing 30 and diaphragm 20.

As shown in FIGS. 2 and 3, the diaphragm 20 may be disposed in tension between biocompatible first and second clamp rings 34a and 34b (e.g. titanium-based) which are interconnected (e.g., via laser welding). In turn, the second clamp ring 34b may be interconnected to a biocompatible cup-shaped bottom member 36 (e.g., via laser welding), wherein the first and second clamp rings 34a, 34b and bottom member 36 combinatively define the housing 30.

A third portion 46 of the enclosed volume 40 may be utilized to house additional componentry of the implantable microphone 1, including for example electronic componentry for generating and/or conditioning an electret output signal. In this regard, and as shown in FIG. 3, vents 62 may be provided through the mount member 60 to fluidly interconnect the second portion 44 and third portion 46 of the enclosed volume 40, thereby further enhancing performance.

In one method of assembly, the diaphragm 20 may be captured between the first and second clamp rings 34a and 34b upon interconnection therebetween (e.g. via laser welding), and such interconnected sub-assembly may be flipped relative to the orientation shown in FIG. 2. In turn, the second peripheral member 32a may be interconnected to the flipped, second clamp ring 34b via complementary, threading 70 provided on the outer periphery of the second peripheral member 32a and inner periphery of the second clamp ring 34b. More particularly, the second peripheral member 32a may be threadably advanced relative to the second clamp ring 34b. Correspondingly, upon such advancement the second peripheral member 32a may progressively contact diaphragm 20 about a ring portion 33 of the second peripheral member 32a to thereby establish a desired degree of tension across the diaphragm 20.

At some point in the assembly process, the assembled electret member 10 may be located relative to the mount member 60, as shown in FIG. 2, wherein the top portion 14a of the T-shaped member 14 may be conformally received by a recessed portion defined on a top surface of the mount member 60. In turn, with electret member 10 and mount member 60 oriented in the position shown in FIG. 2, the locking member 18 may be secured on to the bottom leg portion 14b of the T-shaped member 14 to define an interconnected sub-assembly.

In turn, such interconnected subassembly may be flipped and located relative to the flipped sub-assembly comprising the interconnected first and second clamp rings 34a and 34b, diaphragm 20 and second peripheral member 32a. As may be appreciated, such an approach facilates positioning of the electret member 10 free from user contact with the dielectric material layer 12 of the electret member 10. After flipped positioning of the electret member 10, the first peripheral member 32b may be positioned to capture the mount 60 between the first peripheral member 32b and second peripheral member 32a. More particularly, complimentary threading 72 on the outer periphery of first peripheral member 32b and internal periphery of the second clamp ring 34b may be provided, wherein the first peripheral member 32b may be threadably advanced relative to the second clamp ring 34b so as to securely capture an outer annular portion 62 provided on the mount member 60. Subsequently, after disposing any desired additional componentry within the third portion 46 of the cup-shaped bottom 36, the top member 34 comprising peripheral members 34a and 34b, and the various componentry interconnected thereto described above, may be interconnected to the bottom member 36 (e.g. via laser welding).

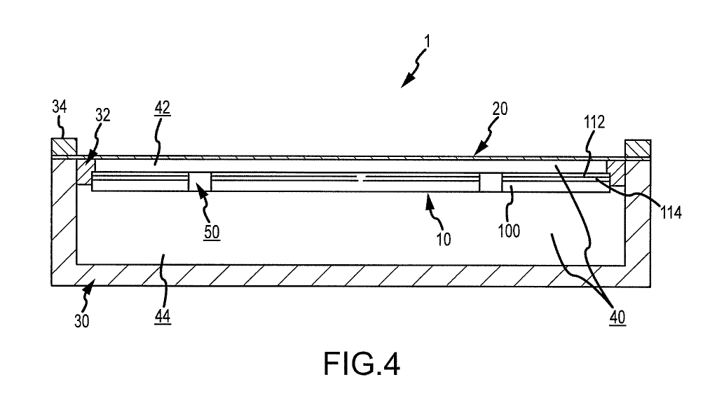

Referring now to FIG. 4, an alternative approach for defining an electret member 10, will be described. In particular, an electrically non-conductive support member 100 (e.g. a printed circuit board) may be provided. In turn, an electrically-conductive, metallized layer may be disposed thereupon to define electrode 114, and in turn, a dielectric coating layer 112 may be disposed thereupon in a viscous or particulate state and dried/cured. For example, the dielectric material may be applied to a desired thickness via dipping, spraying, spin-coating, chemical vapor deposition and/or sputtering. In the later regard, RF sputtering may be employed to simultaneously apply and charge the dielectric material. Alternatively, the dielectric layer 112 may be charged as described hereinabove. As may be appreciated, in the embodiment of FIG. 4 a printed circuit board that defines support member 100 may also be utilized to support various signal processing and other componentry.

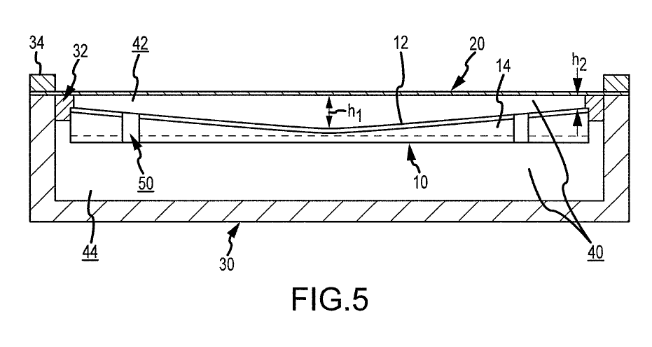

Reference is now made to FIG. 5, in which another embodiment generally corresponding with the embodiment of FIG. 1 is illustrated, wherein corresponding components are referred to with corresponding reference numerals. In the embodiment of FIG. 5, the electret member 10 is shaped so that the top portion 42 of the enclosed volume 40 varies across the lateral extent of the first portion 42. That is, the diaphragm 20 is spaced from a top surface of the dielectric layer by a distance h1 in the middle of the first portion 42 and tapers down to a lesser second distance h2 at an outer periphery of the first portion 42. In turn, greater sensitivity and a relatively flat frequency response may be realized during operation. In the illustrated embodiment, the electrode 14 is shaped to define a shallow-dished or, conic surface upon which the dielectric layer 12 is disposed (e.g., to yield a shallow V-shaped configuration in a the illustrated cross-sectional view of FIG. 5). The electrode 14 may be formed by any of a number of approaches, including for example electro-discharge manufacturing. Alternatively, in another approach, a dielectric layer 12a may be disposed in varying thickness across a uniform thickness electrode 14a to yield a shaped first portion 42 (e.g. via controlled RF sputtering) as shown via phantom lines in FIG. 5.

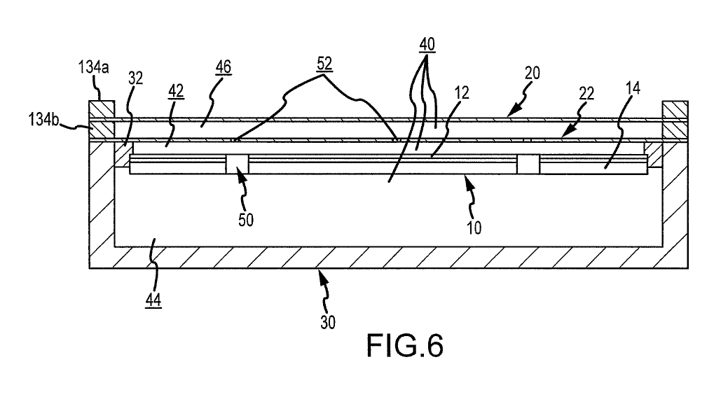

FIG. 6 illustrates yet another embodiment corresponding in part with the embodiment of FIG. 1, wherein corresponding components are referred to with corresponding reference numerals. In the embodiment of FIG. 6, a flexible, electrically conductive back plate 22 may be defined separately from the diaphragm 20 (e.g. the back plate 22 may comprise titanium of an aluminized Mylar). More particularly, and as shown, back plate 22 may be spaced from the diaphragm 20, wherein internal acoustic signals generated by diaphragm 20 will be incident upon the flexible back plate 22. In turn, the flexible back plate 22 may generate internal acoustic signals within the first portion 42 of the enclosed volume 40. As shown, clamp rings 134a, 134b and peripheral member 32 may be provided to dispose the diaphragm 20 and flexible back plate 22 in tension, respectively. Further, peripheral member 32 may comprise an electrically non-conductive material to electrically isolate the back plate 22 and electret member 10. Vents 52 may be provided through the flexible back plate 22 to fluidly interconnect the first portion 42 of the enclosed volume 40 with a third portion 46 located between the diaphragm 20 and flexible back plate 22.

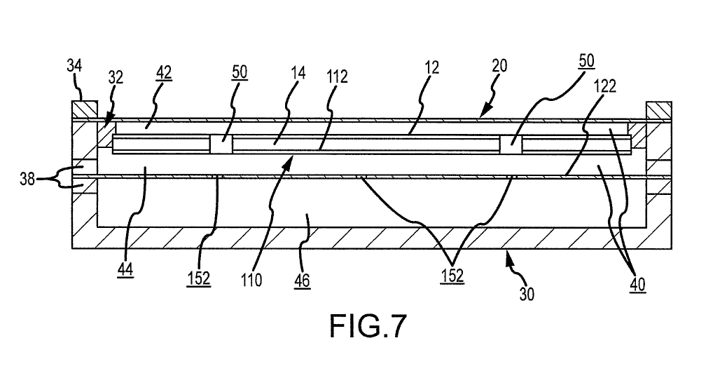

Referring now to FIG. 7, a further embodiment is illustrated, wherein components that correspond with components in the embodiment of FIG. 1 are referred to with corresponding reference numerals. As shown, in the implantable microphone of FIG. 7, a non-flexible electret member 110 is provided having a first dielectric layer 12 disposed on a top, first side of an electrode 14 and a second dielectric layer 112 disposed on a bottom, second side of the electrode 14. In turn, in addition to a diaphragm 20 defining a back plate that opposes the first dielectric layer 12, the illustrated embodiment includes a separate flexible back plate 122 disposed in opposing relation to the second dielectric layer 112. Vents 152 may be provided through the back plate 122 to fluidly interconnect the second portion 44 of the enclosed volume with a third portion 46. Electrically non-conductive members 38 may isolate the back plate 122. The electrical outputs from the back plate electrode of diaphragm 20, the electrode of back plate 122 and electrode 14 of the electret member 110 may be combinatively utilized to provide an electret output signal.

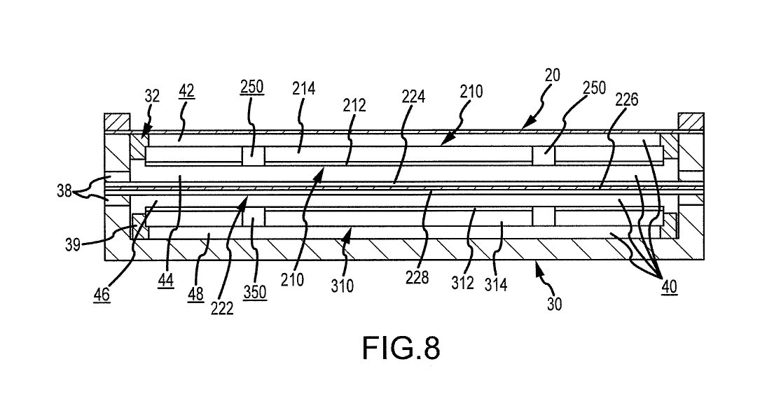

FIG. 8 illustrates yet another embodiment, wherein components that correspond with components in the embodiment of FIG. 1 are referred to with corresponding reference numerals. In this embodiment, an upper, non-flexible electret member 210 and a lower, non-flexible electret member 310 are provided with a flexible back plate 222 disposed therebetween. More particularly, the upper electret member 210 may include a dielectric layer 212 disposed on a bottom side of an electrode 214, and the lower electret member 310 may include a dielectric layer 312 disposed on a top side of an electrode 314. In turn, the flexible back plate 222 may comprise an electrically-conductive electrode 224 disposed on a top first side of a flexible substrate 226 and an electrically-conductive electrode 228 disposed on a bottom side of the flexible substrate 226. By way of example, electrodes 224 and 228 may be disposed via metallization on to the flexible substrate 226 (e.g., a Mylar substrate). A plurality of vents 250 may extend through the upper electret member 210 to fluidly interconnect a first portion 42 and second portion 44 of the enclosed volume. Similarly, a plurality of vents 350 may extend through the lower electret member 310 to fluidly interconnect a third portion 46 and forth portion 48 of the enclosed volume 40. As may be appreciated, the double-electrode-sided back plate 222 may be electrically isolated via insulator members 38, and the lower electret member 310 may be electrically isolated via support/insulator members 39. In the illustrated arrangement, the electrical outputs from electrode 214 and electrode 224, as well as the electrical outputs from electrode 228 and electrode 314, may be combinatively employed to generate the electret output signal.

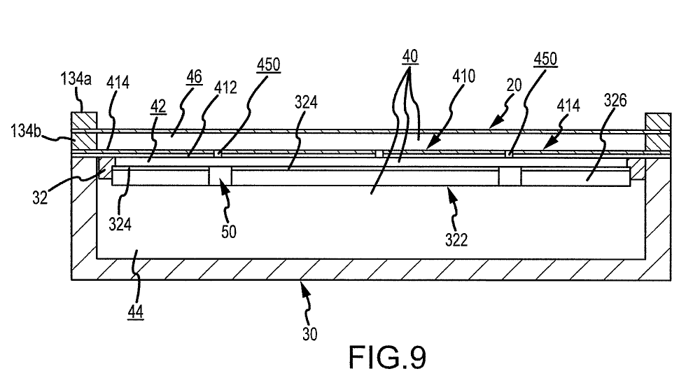

Referring now to FIG. 9, another embodiment is illustrated, wherein components corresponding with those referred to in the embodiment of FIG. 1 utilize corresponding reference numerals. In this embodiment, a flexible electret member 410 is disposed in spaced relation below flexible diaphragm 20 and above a non-flexible back plate 322. As shown, the flexible electret member 410 may include a dielectric layer 412 disposed on a bottom side of an electrode 414. The back plate 322 may include an electrically-conductive electrode 324 disposed (e.g. via metallization) on a top surface of an electrically non-conductive substrate 326 (e.g., a printed circuit board). A shown, a plurality of vents 50 may extend through the back plate 322 to fluidly interconnect a first portion 42 of an enclosed volume 40 with a second portion 44 of the enclosed volume 40. Further, a plurality of vents 450 may extend through the electret member 410 to fluidly interconnect a third portion 46 of the enclosed volume 40 with a first portion 42 thereof.

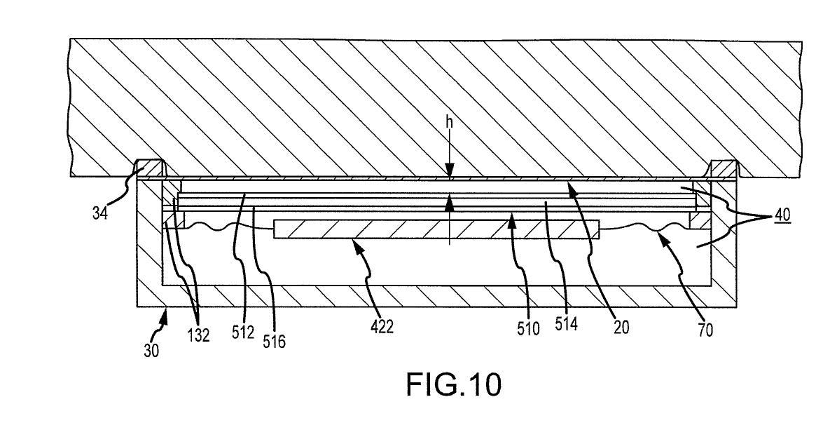

Reference is now made to FIG. 10 which illustrates yet another embodiment, wherein components corresponding with the components of the FIG. 1 embodiment are referenced with corresponding reference numerals. In this embodiment, a non-flexible electret member 510 includes a first dielectric layer 512 disposed on a top side of electrode 514 and a second dielectric layer 516 disposed on a bottom side of electrode 514. A flexible outer diaphragm 20 may define a first back plate located in spaced relation to the first dielectric layer 512 of the electret member 510. A second back plate 422 may be disposed in spaced opposing relation to the second dielectric layer 516 of the electret member 510. In this regard, the second back plate 422 may be supported via one or more flexible members 70. Electrical isolation and support of electret member 510 is provided by members 132. As may be appreciated, electrical outputs from the first back plate of diaphragm 20, the electrode 514 of the electret member 510, and the second back plate 522 may be combinatively utilized to provide an electret output signal.

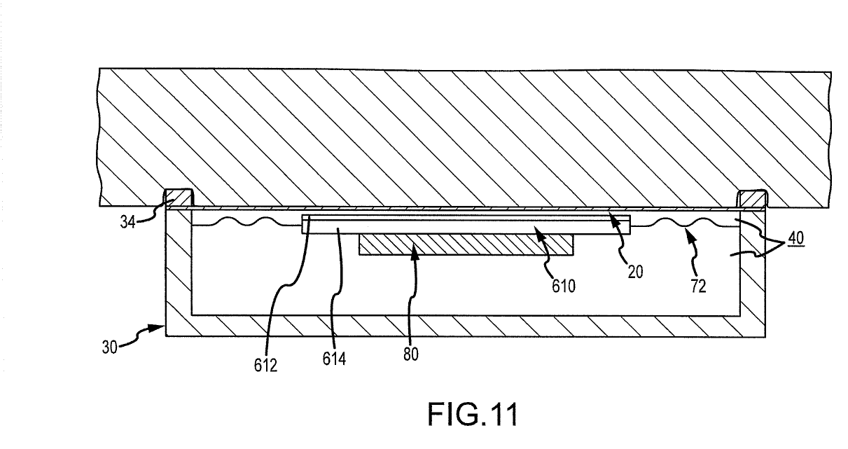

Reference is now made to FIG. 11 which illustrates an additional embodiment, wherein components corresponding with components of the embodiment of FIG. 1 are referred to with corresponding reference numerals. As shown, the flexible electret member 610 may include a dielectric layer 612 disposed on an electrode 614, wherein the electret member 610 is suspended via one or more flexible members 72. The electret member 610 may have a proof mass 80 interconnected thereto. The flexible diaphragm 20 may define a back plate located in spaced relation to the electret member 610.

Various modifications and other embodiments to those described hereinabove will be apparent to those skilled in the art and are intended to be within the scope of the present invention.

What is claimed is:

1. An implantable microphone comprising:
a hermetically-sealed, enclosed volume;
an electret member and a back plate disposed with a space therebetween and capacitively coupleable to provide an output signal indicative of acoustic signals incident upon at least one of the electret member and back plate, said space being within said enclosed volume, wherein at least a first portion of said enclosed volume is located on a first side of said electret member and at least a second portion of said enclosed volume is located on a second side of said electret member; and
at least one vent interconnecting said first and second portions.

2. The microphone of claim 1, wherein said back plate defines at least a peripheral portion of said enclosed volume.

3. The microphone of claim 2, wherein said back plate one of defines and is interconnected to a flexible diaphragm for receiving external acoustic signals and generating internal acoustic signals within said enclosed volume in response thereto.

4. The microphone of claim 1, wherein said at least one vent extends through said electret member.

5. The microphone of claim 4, further comprising:
a plurality of vents interconnecting said first and second portions and extending through said electret member.

6. The microphone of claim 5, wherein said plurality of vents are spaced in a symmetric manner about a center axis of said electret member.

7. An implantable microphone comprising:
a hermetically-sealed, enclosed volume;
a flexible, biocompatible diaphragm defining a peripheral portion of said enclosed volume;
a flexible electret member and a back plate disposed with a space therebetween and capacitively coupleable to provide an output signal indicative of acoustic signals incident upon said flexible electret member, said space being within a first portion of said enclosed volume, wherein said first portion of said enclosed volume is located on a first side of said back plate and a second portion of said enclosed volume is located on a second side of said back plate; and
and at least one vent interconnecting said first and second portions extending through said back plate.

8. The microphone of claim 7, further comprising:
a plurality of vents interconnecting said first and second portions and extending through said electret member.

9. The microphone of claim 8, wherein said plurality of vents are spaced in a symmetric manner about a center axis of said electret member.

10. An implantable microphone comprising:
a hermetically-sealed, enclosed volume;
an electret member and back plate disposed with a space therebetween and capacitively comparable to provide an output signal indicative of acoustic signals incident upon at least one of the electret member and back plate, said space being within said enclosed volume, wherein one of said electret member and back plate comprises:
a support member having a layer of material applied thereto in one of a viscous state and a particulate state, said applied material being one of cured and dried upon said support member, wherein said material is supportably disposed on said support member.

11. The microphone of claim 10, wherein one of said electret member and said back plate one of defines and comprises a flexible diaphragm for receiving external acoustic signals.

12. The microphone of claim 10, wherein said electret member comprises said support member, and wherein said layer of material comprises an electrically conductive material.

13. The microphone of claim 12, further comprising:
a layer of dielectric material applied to one of said support member and said layer of electrically conductive material in one of a viscous state and a particulate state, said dielectric material being one of dried and cured.

14. The microphone of claim 13, wherein said space is within a first portion of said enclosed volume, wherein said first portion of said enclosed volume is located on a first side of said electret member and a second portion of said enclosed volume is located on a second side of said electret member, said microphone further comprising:
at least one vent interconnecting said first and second portions and extending through said electret member.

15. The microphone of claim 10, wherein said electret member comprises said support member, and wherein said layer of material comprises a dielectric material.

16. The microphone of claim 15, wherein said layer of dielectric material is of varying thickness across a lateral extent thereof and defines a varying distance between said electret member and said back plate across a lateral extent of said space therebetween.

17. The microphone of claim 15, wherein said dielectric material is permanently charged upon application to said support member.

18. The microphone of claim 15, wherein said support member is electrically conductive.

19. The microphone of claim 15, further comprising:
a plurality of vents extending through said electret member.

20. An implantable microphone comprising:
a hermetically-sealed, enclosed volume;
a first electret member and first back plate disposed with a space therebetween and capacitively coupleable to provide an output signal indicative of acoustic signals incident upon at least one of the electret member and back plate, said space being within a first portion of said enclosed volume, wherein said first portion of said enclosed volume is located on a first side of said first electret member and at least a second portion of said enclosed volume is located on a second side of said electret member; and
a second electret member and a second back plate disposed within a space therebetween, said space being within said second portion of said enclosed volume.

21. The microphone of claim 20, wherein one of said first electret member and said first back plate and one of said second electret member and said second back plate are located on opposing sides of a carrier member.

22. The microphone of claim 21, wherein said one of said first electret member and said first back plate, and said one of said second electret member and said second back plate are each defined by metallized layer portions on said carrier member.

23. The microphone of claim 22, wherein said carrier member is flexible.

24. The microphone of claim 23, wherein said carrier member comprises a Mylar sheet.

25. The microphone of claim 20, wherein said first electret member and said second electret member each comprise portions of corresponding metallized layers disposed on opposing sides of a flexible carrier member.

26. The microphone of claim 20, wherein said first back plate defines at least a peripheral portion of said enclosed volume.

27. The microphone of claim 26, wherein said first back plate one of defines and is interconnected to a flexible diaphragm for receiving external acoustic signals and generating internal acoustic signals within said enclosed volume in response thereto.

28. The microphone of claim 26, wherein said first back plate defines a flexible diaphragm for receiving external acoustic signals and generating internal acoustic signals within said enclosed volume in response thereto, and wherein said first back plate comprises a biocompatible, electrically-conductive material.

29. The microphone of claim 26, wherein said first back plate is interconnected to a flexible diaphragm for receiving external acoustic signals and generating internal acoustic signals within said enclosed volume in response thereto, and further comprising:
an electrically non-conductive, insulator interposed between said first back plate and said flexible diaphragm.

30. An implantable microphone comprising:
a hermetically-sealed, enclosed volume; and
an electret member and a back plate disposed with a space therebetween and capacitively coupleable to provide an output signal indicative of acoustic signals incident upon at least one of the electret member and back plate, said space being within said enclosed volume, wherein said back plate defines at least a peripheral portion of said enclosed volume.

31. The microphone of claim 30, wherein said back plate one of defines and comprises a flexible diaphragm for receiving external acoustic signals and generating internal acoustic signals within said enclosed volume in response thereto.
